(19) United States
(12) Patent Application Publication
(10) Pub. No.: US 2004/0154622 A1
(43) Pub. Date: Aug. 12, 2004

(54) Endotracheal tube and an apparatus, and method of making same, for securing an endotracheal tube

(76) Inventor: Billy B. Davis, Fort worth, TX (US)

Correspondence Address: DECKER, JONES, MCMACKIN, MCCLANE, HALL &, BATES, P.C., BURNETT PLAZA 2000, 801 CHERRY STREET, UNIT #46, FORT WORTH, TX, 76102-6836, US

(21) Appl. No.: 10/361,306

(22) Filed: Feb. 10, 2003

(51) Int. Cl. A62B 9/06; A61M 16/00

(52) U.S. Cl. 128/207.14; 128/207.17

(57) ABSTRACT

A tracheal tube, having a machine end and a patient end, includes a tracheal tube tie located between the machine and patient ends of the tube. The tracheal tube tie is elastic, is made from twisted plastic tape that has been heated to make a rounded cord, and has loose ends for securing the tube in a trachea of a patient by tying the ends to the nose or jaw of a veterinary patient. The plastic tape may be made of polyvinyl chloride. The tape is twisted along its length to form a rounded cord. The cord is heat treated so that it keeps the rounded form.

### BACKGROUND OF THE INVENTION

1. Field of the Invention

The present invention relates generally to medical devices, and more specifically to an endotracheal tube and a tracheal tube tie made from a plastic tape material for securing the tracheal tube.

2. Description of the Prior Art

When a veterinarian operates on a patient, such as a dog, an air tube is often needed to assist in breathing or administering medication. For example, a mixture of oxygen and anesthetic gas may be provided through a endotracheal tube—a tube that is inserted into a patient's trachea, or windpipe—during a medical procedure, such as cleaning a dog's teeth, or removing decaying teeth, or any type of surgery. This allows the procedure to be pain-free, and the level of anesthesia administered is safer because it is easier to regulate.

An endotracheal tube is typically made from clear plastic, such as silicone, that is rigid enough to be guided during insertion, but not so rigid that it will not bend to conform to a curve of the trachea. To accommodate patients of different sizes, the inside diameter of an endotracheal tube may range from 3 millimeters (mm) to 14 mm.

Once the endotracheal tube is inserted, it should be secured so that it does not come out, and so that its movement relative to the trachea is limited to reduce irritation. Laryngeal and tracheal tissue damage may occur if the tube moves too much. In the prior art, the endotracheal tube was tied to the patient using roll gauze. The gauze was first tied tightly around the tube using a half-hitch. This first tie must be tight enough to prevent slippage, but not tight enough to collapse the tube.

After tying gauze around the tube, the gauze can then be tied around the upper jaw or lower jaw, behind the canine teeth, or tied behind the ears, or neck, in animals that do not have a muzzle. The tongue should not be entrapped, and circulation to the nose or jaw should not be compromised.

Difficulties with the prior art gauze include the inelasticity of gauze, which tends to roll or bunch and can cause restriction of blood flow in the nose or jaw. Also, because gauze has many openings in the weave, gauze is likely to become tangled and knotted with fur, making tying and untying the gauze more difficult. In addition, gauze tends to slip along the endotracheal tube allowing tracheal damage, premature extubation, and possible patient death from suffocation.

Another prior art tie involves the use of string, which is likewise inelastic and can cut into the muzzle or neck. Also, the string can be difficult to find once it is tied onto a long-haired animal.

Both gauze and string have a low coefficient of friction relative to the endotracheal tube's external surface.

Therefore, a need exists for an improved endotracheal tube for veterinary patients, wherein a tie for securing the endotracheal tube is inexpensive, easy to tie, easy to see, and is not unduly restrictive to blood flow in the patient. Such tie should also have a higher coefficient of friction relative to the endotracheal tube's external surface to prevent slippage of the tube within the patient's trachea. There also exists a need for an improved tracheal tube tie, and an improved method for making the tracheal tube tie.

### SUMMARY OF THE INVENTION

The foregoing problems are overcome and other advantages are provided by a tracheal tube having a tracheal tube tie of the invention.

In accordance with an embodiment of the invention, a tracheal tube, having a machine end and a patient end, includes a tracheal tube tie located between the machine and patient ends of the tube. The tracheal tube tie is elastic, is made from twisted plastic tape that has been heated to make a rounded cord, and has loose ends for securing the tube in a trachea of a patient by tying the ends to the nose or jaw of a veterinary patient. The plastic tape may be fluorescent tape made of polyvinyl chloride. The tape is twisted along its length to form a rounded cord. The cord is heat and pressure treated so that it keeps the rounded form.

An endotracheal tube assembly of the present invention includes an endotracheal tube having a machine end and a patient end. A plastic tube tie is coupled to the endotracheal tube between the machine end and the patient end, wherein the plastic tube tie is elastic, made from twisted plastic tape that has been heated to make a cord, and has loose ends for tying to a veterinary patient. The plastic tape may be polyvinyl chloride fluorescent tape. The twisted plastic tape has been heated to set the cord shape at a temperature of between 200° F.-375° F. for 20 minutes to one hour at 10-15 lbs/in² of pressure.

A method of making a tie for a tracheal tube includes twisting a flat plastic tape to form a rounded cord, and heating and pressure treating the rounded cord to set the round form. The rounded cord may be on a spool and heated in an oven, such as an autoclave. Alternatively, the rounded cord may be heated as it passes through an oven in a continuous feed. The heating may be between 200° F. and 375° F. for 20 minutes to 1 hour.

### BRIEF DESCRIPTION OF THE DRAWINGS

For a more complete understanding of the present invention, and the advantages thereof, reference is now made to the following descriptions taken in conjunction with the accompanying drawings, in which like numbers designate like parts, and in which:

FIGS. 1 and 2 are side views of an animal patient, such as a dog, using the tracheal tube and tracheal tube tie in accordance with embodiments of the present invention;

FIG. 3 is a side view of a tracheal tube having a tracheal tube tie in accordance with an embodiment of the present invention; and

FIG. 4 is a schematic representation of methods of making a tracheal tube tie in accordance with the present invention.

### DESCRIPTION OF THE PREFERRED EMBODIMENTS

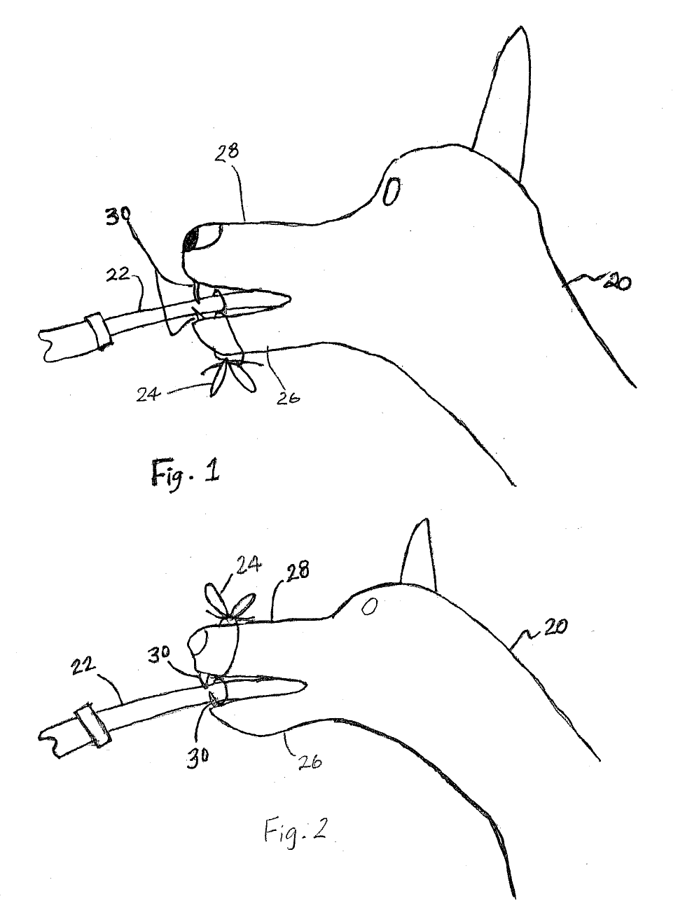

With reference now to the drawings, and in particular with reference to FIGS. 1 and 2, there is depicted a side view of a veterinary patient—which in these figures is a dog—having a tracheal tube inserted into the trachea, and the tracheal tube secured by a tie in accordance with the present invention. As shown, patient 20 (or dog 20) is lying on its side with its mouth open and tracheal tube 22 inserted into the patient's trachea, or wind pipe. Patient 20 may be a dog, cat, pig, or other animal with an extended or projecting nose and jaw, such as the muzzle or snout of a dog. The invention can be used on small animals as well as large animals, such as horses, cattle, etc. The invention can also be used on animals lacking a muzzle or snout.

To prevent tissue damage due to tracheal tube movement, and to prevent the dislocation of the tracheal tube, tracheal tube 22 is secured to patient 20 by tying it with tracheal tube tie 24, which is tied to both tracheal tube 22 and patient 20. As illustrated in FIG. 1, tracheal tube tie 24 may be tied to the patient's 20 jaw 26. Or, as shown in FIG. 2, tracheal tube tie 24 may be tied to the patient's 20 nose 28. In either case, tracheal tube tie 24 is typically tied behind canine teeth 30. If patient 20 has no snout, or a short snout, the tracheal tube tie may alternatively be tied behind the patient's ears, on the neck.

Tracheal tube tie 24 is preferably tied to tracheal tube 22 with a knot such as a square knot or half-hitch, leaving long, loose ends that can be tied around the nose or jaw of patient 20.

As described in more detail below, tracheal tube tie 24 is elastic so that it can be stretched and then return to its original length. This elasticity is desirable in that the tie 24 can be firmly and snuggly tied to securely hold the tube 22, yet prevent excessive restriction of blood flow in the patient's nose or jaw. When the elasticity allows some give or yield, blood in the nose or jaw can circulate past tracheal tube tie 24. The tie 24 will hold securely to the tube 22. Likewise the tie will hold a knot.

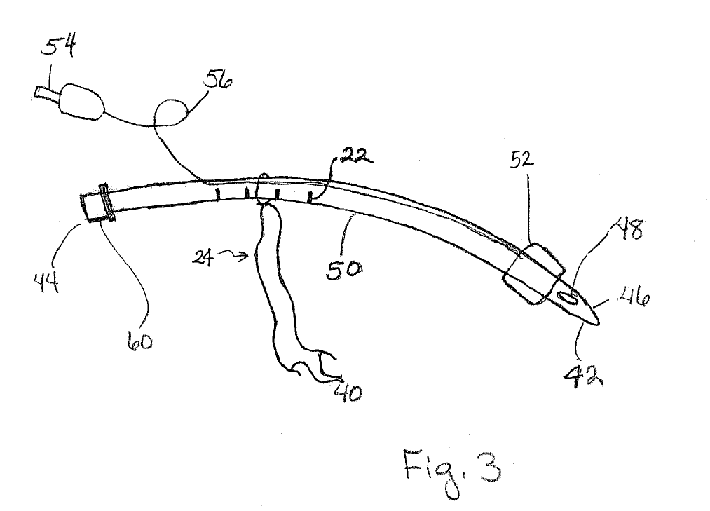

FIG. 3 is a side view of a conventional tracheal tube having a tracheal tube tie 24 in accordance with an embodiment of the present invention. As depicted, tracheal tube 22 includes tracheal tube tie 24, which is attached by a knot; other methods of attachment could be used such as by a PVC welding technique, namely a heat or chemical weld. Tracheal tube tie 24 has loose ends 40 that are long enough to tie around the nose or jaw (or the neck) of a patient.

Tracheal tube 22 has a patient end 42, which is inserted into the patient's mouth and trachea, and a machine end 44, which is connected by a hose to a breathing machine, which machine may administer oxygen and anesthesia. Bevel 46 on patient end 42 helps to guide the tube. Murphy eye 48 is an opening on patient end 42 for air and gas to enter and leave main tube 50. Cuff 52 is an air bladder that is inflated using inflation valve 54 and inflation tube 56, which runs down the side of the tube and into cuff 54. Inflating cuff 52 seals the space between the tube and the tracheal wall. Graduation marks on the side of main tube 50 are a guide for how deep the tube has been inserted. Machine end 44 has connector 60 for coupling to a hose that comes from the breathing machine.

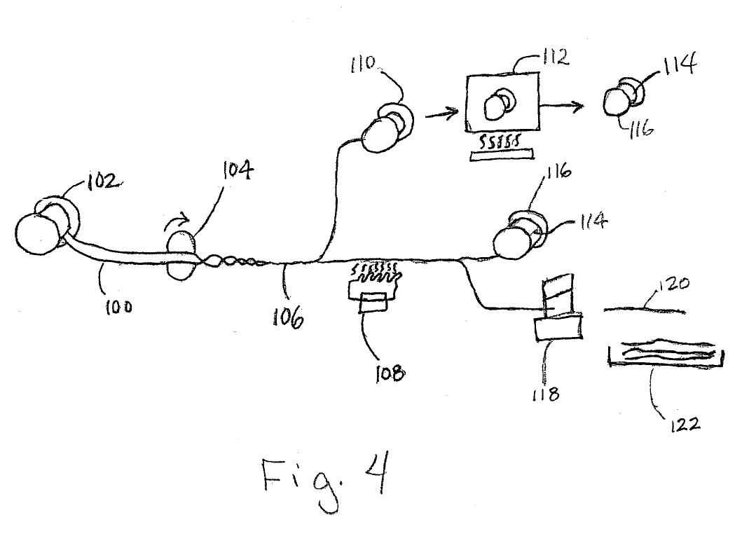

Tracheal tube tie 24 is preferably made from high-quality polyvinyl chloride (PVC) by a process that is illustrated in FIG. 4, although other types of plastics may work satisfactorily. As shown in FIG. 4, fabrication begins with providing a plastic tape, such as PVC tape 100, which may be flagging tape used by surveyors to mark property boundaries. The plastic tape should have a high tensile strength, and be elastic, so that it returns substantially to its original length after being stretched. In a preferred embodiment, PVC tape 100 is about 1 inch wide and about 2 mils (thousandths of an inch) thick, and is preferably smooth and not embossed with a pattern. The PVC tape can typically be greatly elongated, from over twice its length to as much as 270% of its original length. Using a brightly colored or fluorescent tape (such as orange) is also an advantage because the tracheal tube tie may be easily located when it is tied in or through the midst of animal fur. The tape may be purchased in rolls or spools 102.

The process begins with twisting the PVC tape along its length to form the flat tape into a round cord 106, which cord is flexible and elastic, similar to the tape. The twisting is preferably done with a machine 104. In a simple process, a length of PVC tape 100 may be secured on one end and attached to a drill, or other rotary device, on the other end. The drill may be used to twist the length of tape until it is rounded and relatively tightly wrapped upon itself so that it is about the diameter of a shoe lace, which diameter is preferably between 1-5 millimeters (mm). In a process for efficiently making larger quantities of twisted tape, a twisting machine 104 may be used as part of a batch process that continually feeds tape into twisting machine 104. Twisting machine 104 may also be a machine that holds and turns the spool as it simultaneously dispenses and twists PVC tape 100.

After forming the tape into cord 106, the cord is heat treated so that it bonds or sets with heat into the round cord shape, wherein it resists becoming untwisted and returning to flat tape. In a preferred embodiment, cord 106 is wrapped around a spool 110, and then placed into a standard pressure-cooker (such as an autoclave) 112. The cord is heated to a temperature of between 200° F.-375° F. for a period of time. The amount of time depends on the temperature, but ranges between 20 minutes to one hour. In the preferred embodiment, the cord is autoclaved for 20 minutes at a temperature between 210° F.-300° F. (and preferably at 250° F.) and a pressure of 5-20 psi. This produces a rounded cord that will not unravel and has a desired coefficient of friction, wherein the cord will not slip on the tube 22. It is believed that heating the cord without pressure treatment will produce a cord with somewhat similar characteristics. As shown in FIG. 4, alternatively, cord 106 may be heated in a continuous process by heater or oven 108. The process sets the rounded form, preventing the unwinding of the tape. In the preferred embodiment, a pressure of 10-15 psi is used.

After spool 110 cools, the tracheal tube tie cord 114 may be distributed and sold on take-up spool 116, so that a veterinarian can unroll and cut a desired length as needed in a medical operation. Alternatively, cord 106 may be cut by cutter 118, and lengths of tracheal tube ties 120 may be collected in bin 122 for packaging and distribution.

The foregoing description of a preferred embodiment of the invention has been presented for the purpose of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An endotracheal tube assembly comprising:
an endotracheal tube having a machine end and a patient end; and
a plastic tube tie coupled to the endotracheal tube between the machine end and the patient end, wherein the plastic tube tie is elastic, made from twisted plastic tape that has been heated to make a cord, and has loose ends for tying to a veterinary patient.

2. The endotracheal tube according to claim 1 wherein the plastic tape is polyvinyl chloride.

3. The endotracheal tube according to claim 1 wherein the plastic tape is flagging tape.

4. The endotracheal tube according to claim 1 wherein the plastic tape is fluorescent tape.

5. The endotracheal tube according to claim 1 wherein the plastic tape has been heated to between 200° F.-375° F. for 20 minutes to one hour.

6. A tie for a tracheal tube comprising:
a plastic tape that has been twisted to form a cord and heated to set a round cord shape.

7. The tie for a tracheal tube according to claim 6 wherein the plastic tape is P.V.C. tape.

8. The tie for a tracheal tube according to claim 6 wherein the plastic tape is florescent tape.

9. The tie for a tracheal tube according to claim 6 wherein the heating is from 200° F.-375° F. for 20 minutes to one hour.

10. The tie for a tracheal tube according to claim 6 wherein the twisting forms a cord from 1 to 5 millimeters in diameter, wherein the cord is formed of flat tape wrapped around itself.

11. The tie for a tracheal tube according to claim 6 wherein the twisted tape is pressure treated as well as heated.

12. A method of making a tie for a tracheal tube comprising the steps of:
twisting a flat plastic tape to form a rounded cord; and
heating the rounded cord to set the round form.

13. The method of making a tie for a tracheal tube according to claim 12 wherein the step of heating the rounded cord to set the round form further includes the steps of rolling the rounded cord on a spool and heating the spool in an oven.

14. The method of making a tie for a tracheal tube according to claim 12 wherein the step of heating the cord to set the rounded form further includes the step of heating the rounded cord as it passes through an oven in a continuous feed.

15. The method of making a tie for a tracheal tube according to claim 12 wherein the step of twisting a flat plastic tape to form a rounded cord further includes twisting a flat plastic polyvinyl chloride flagging tape to form a rounded cord from 1 to 5 millimeters in diameter.

16. The method of making a tie for a tracheal tube according to claim 12 wherein the step of heating the rounded cord to set the round form further includes heating the rounded cord to between 200° F. and 375° F. for 20 minutes to 1 hour to set the round form.
